(19) United States
(12) Patent Application Publication
(10) Pub. No.: US 2022/0054781 A1
(43) Pub. Date: Feb. 24, 2022

(54) A SPEECH VALVE

(71) Applicant: KAPITEX HEALTHCARE LIMITED, Yorkshire (GB)

(72) Inventors: Ian David WORTHINGTON, Yorkshire (GB); Alexander James BRIERLEY, Yorkshire (GB)

(21) Appl. No.: 17/298,747

(22) PCT Filed: Jan. 14, 2020
(86) PCT No.: PCT/EP2020/050838

§ 371 Date: Jun. 1, 2021

(30) Foreign Application Priority Data
Jan. 14, 2019 (GB) 1900490.2

(51) Int. Cl. A61M 16/04; A61F 2/20

(52) U.S. Cl. CPC A61M 16/0468; A61F 2002/206; A61F 2/20

(57) ABSTRACT

A speaking valve for use with a tracheostomy and voice prosthesis having a valve element movable by finger pressure between a first position in which the valve allows air to pass through it and a second position in which the passage of air is substantially resisted or prevented. A biasing element such as foam or a spring is provided to bias the valve element towards the first position when no finger pressure is applied. The valve element is arranged such that expiration through the valve at a rate in excess of predetermined rate causes it to move into a third position in which air resistance through the valve is less than when the valve element is in its first position. This allows for air release if e.g. a user should cough.

### FIELD OF THE INVENTION

The invention relates to speaking valves, used to control the flow of air through a tracheostoma, and also to valve elements suitable for use in such speaking valves and elsewhere, especially in medical devices, and more particularly in medical airway management devices.

### BACKGROUND AND PRIOR ART

Speaking valves are known in themselves, and are used to control, or assist the control of the flow of air through a tracheostoma—a passage formed between the trachea of a human subject, and the outside air.

One particular, and typical use, will be described with reference to FIGS. 1 and 2. FIG. 1 shows a schematic cross-section through a human subject 1 who has undergone a laryngectomy. FIGS. 2A and 2B illustrate an enlargement of the region enclosed by the dashed-line circle. The removal of the larynx is often a consequence of throat cancer. Illustrated are the trachea 2, or windpipe, connecting the lungs to the tracheostoma 3, and also the oesophagus 4 connecting the stomach to the mouth 5. In order to allow a person to speak, in the absence of a larynx, a voice prosthesis 6 is fitted within a fistula made between the trachea 2 and the oesophagus 6. The voice prosthesis acts as a one-way valve, preventing food and drink within the oesophagus 6 reaching the lungs via the trachea 2. A speaking valve 7 is provided within the tracheostoma, between the outside air and the opening to the trachea 2. In its non-speaking configuration, the speaking valve 7 allows the flow of air into and out of the trachea as illustrated by the arrow in FIG. 2A, thereby allowing the individual to breathe.

When the individual wishes to speak, they take air into their lungs with the speaking valve 7 in the configuration shown in FIG. 2A, and then cause the valve 7 to close while exhaling the air. With the speaking valve 7 closed, air is forced through the voice prosthesis 6 and into the upper region of the oesophagus as indicated by the arrow in FIG. 2B. This flow of air vibrates soft tissue within the patient's oesophagus causing sound to be produced that the individual can control to produce speech.

In its simplest form, a speaking valve 7 could constitute an aperture through the tracheostoma that the individual can close by covering the aperture with a finger or thumb. In more advanced valves, a valve element occluding the aperture might be provided, biased into an open position. An individual can then push and hold the valve element into a closed position while speaking.

One problem that occurs with such speaking valves is that, if the user coughs, the speaking valve cannot accommodate the increased rate of air flow. In this situation there is a risk of injury to the user or, in the best case, the speaking valve will be discharged from the tracheostoma. This can be very embarrassing for the user.

It is among the objects of the present invention to provide a solution to these and other problems.

### SUMMARY OF THE INVENTION

Accordingly, the invention provides a speaking valve to allow inspiration and controllable expiration through a tracheostoma, said speaking valve comprising: a valve element movable by finger pressure of a user between a first position in which said speaking valve allows air to pass through it and a second position in which the passage of air through said speaking valve is substantially resisted or prevented; a biasing element to bias said valve element towards said first position when no finger pressure is applied; wherein said valve element is arranged such that expiration through the valve at a rate in excess of predetermined rate causes said valve element to move into a third position in which air resistance through the valve is less than when the valve element is in its first position.

Preferably, when said valve element is in its third position it can only be moved to its first position by finger pressure of a user.

In either case, it is also preferred that said biasing element comprises resilient open cell foam.

### BRIEF DESCRIPTION OF THE FIGURES

The invention will be described with reference to the accompanying drawings, in which:

FIGS. 1 and 2 illustrate, in cross-section, the location of a speaking valve and voice prosthesis in a user;

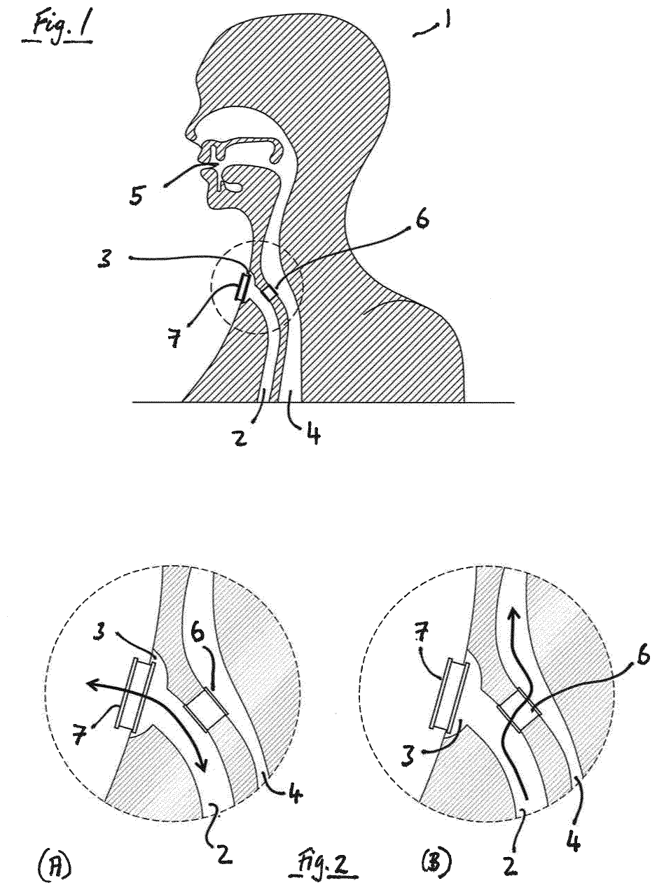

FIG. 3 illustrates, in cross-section, an embodiment of a speaking valve of the invention;

FIG. 4 illustrates, in exploded perspective view, an embodiment of a speaking valve of the invention; and

FIGS. 5-6 illustrate, in cross-section, an embodiment of a speaking valve of the invention.

### DESCRIPTION OF PREFERRED EMBODIMENTS

In this discussion of speaking valves, when we talk of a pressure difference across a valve, or across a diaphragm member of a valve, we shall define the pressure difference as being positive when the pressure on the tracheal side of the valve or diaphragm is greater than the pressure on the other side, i.e. typically atmospheric air pressure.

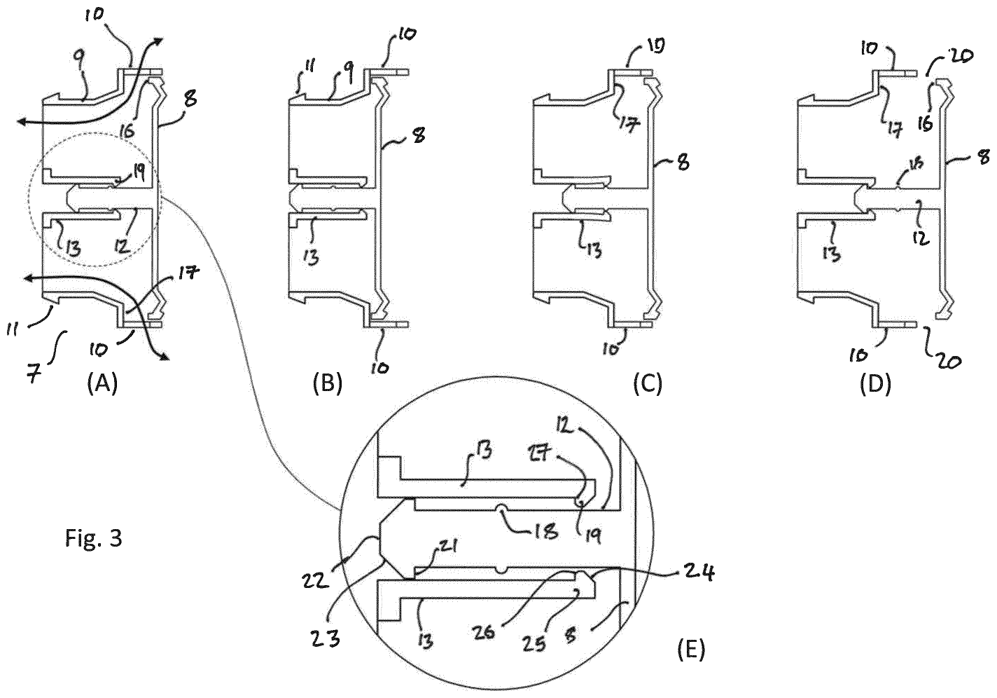

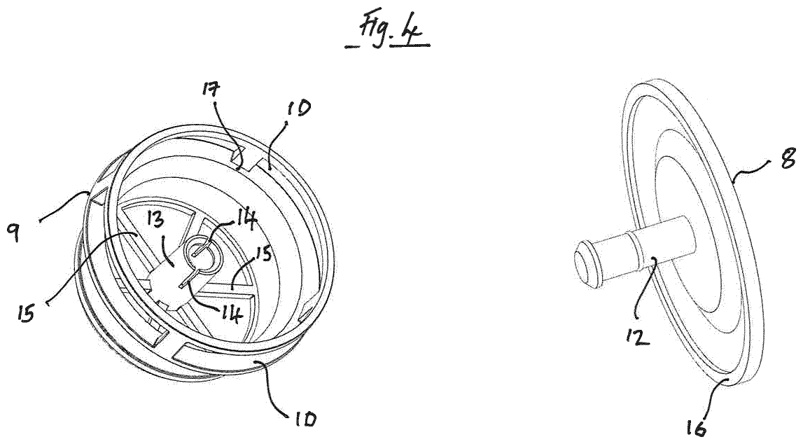

FIG. 3 illustrates, in cross-sectional view an embodiment of a speaking valve of the invention, generally indicated by 7. The speaking valve 7 is of generally cylindrical form, as illustrated in perspective view in FIG. 4. FIGS. 3A-3D illustrate the speaking valve with the valve element 8 in various positions, while FIG. 3E illustrates an enlarged region of FIG. 3A indicated by the dashed circle.

The speaking valve comprises a generally cylindrical valve body 9, preferably provided with an attachment lug or ring 11 to enable it to be connected to a self-adhesive baseplate (not illustrated) that may be attached around a user's tracheostoma. Such baseplates are well-known in the art. The side wall of the valve body 9 is provided with apertures 10 towards the end further from the tracheal side of the valve 7 that allow air to pass through, as illustrated by the arrows in FIG. 3A.

A valve element 8 in the form of a disc is supported on a rod 12 extending from its rear face. The rod 12 is locatable within a hollow cylindrical support 13 forming part of the valve body 9. The support 13 is formed of a resiliently deformable material, such as plastics, and is provided with one or more longitudinal slots 14 to allow the support to open when an outwardly-directed force is applied to the support. These are illustrated in the exploded perspective view of FIG. 4. The support 13 is attached to the side wall of the valve body 9 by a number of radial spokes 15.

FIG. 3A illustrates the speaking valve 7 with the valve element 8 in its first position, allowing air to pass through the valve, via the apertures 10. The valve element 8 is biased into this position by a biasing element (not illustrated) that will be described below. Further movement of the valve element 8 is resisted by the interaction of an outwardly-extending protuberance 18 on the surface of the rod 12 with an inwardly-extending region 19 on the inner face of the support 13. In this embodiment the protuberance 18 is in the form of a raised ring having a curved profile.

When finger pressure is applied on the outer face of the valve element 8 to counteract the biasing force, the valve element 8 moves into its second position, as illustrated in FIG. 3B. In this position, an outer region 16 of the inner face abuts an opposing face 17 on the valve body 9 causing the passage of air through the speaking valve to be substantially resisted or prevented. To achieve this, the abutment between the outer region 16 and the opposing face 17 is located on the tracheal side of the apertures 10. When the finger pressure is removed, the valve element 8 returns to the open position of FIG. 3A.

In the event that the flow rate of air out of the trachea exceeds a predetermined value, for example if the user coughs, the force of the air on the inner face of the valve element 8 causes the valve element 8 to move into its third position. FIG. 3C illustrates how the protuberance 18 interacts with the inwardly-extending region 19 to deform the support 13, allowing the valve element 8 to move into its third position as illustrated in FIG. 3D. In this position, air is now able to flow not only through the apertures 10, but also through the annular opening 20 between the edge of the valve member 8 and the forward face of the valve body 9. In this way, the flow resistance through the valve is reduced to less than that when the valve element 8 is in its first position illustrated in FIG. 3A. In preferred embodiments, the valve element 8 is held in the open position of FIG. 3D by the action of the biasing element, but is held captive to the valve body by the interaction of an outwardly-facing stop portion 21 with the inwardly-extending region 19 on the inner face of the support 13.

It can be seen, most clearly in FIG. 3E, that the interacting faces of the rod 12 and the support 13 are specifically shaped to assist in the operation of the valve, and to facility the valve's assembly. The proximal end 22 of the rod 12 is provided with a bevelled surface 23 that interacts with a correspondingly bevelled surface 24 on the inner edge of the distal end 25 of the support 13. Also, outer surface of the protuberance 18 is rounded, as is the innermost proximal edge 26 of the inwardly-extending region 19. This facilitates a controlled transition from the first position (FIG. 3A) to the third position (FIG. 3D). Finally, the outwardly-facing stop portion 21 and the outermost proximal edge 27 of the inwardly-extending region 19 are each provided with faces perpendicular to the main axis of the valve 7 to provide a firm engagement to prevent the valve element 8 and valve body 9 coming apart once in the third position of FIG. 3D.

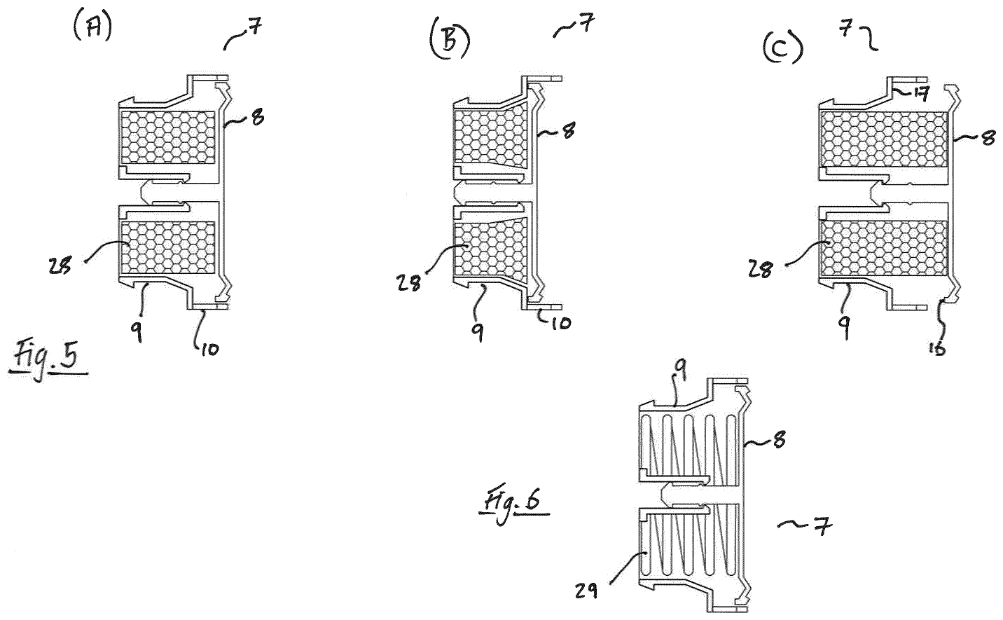

FIGS. 5A-5B illustrate an embodiment of a speaking valve 7 of the invention in cross-sectional view to show the positioning of a biasing element. The illustrated embodiment is the same as that illustrated in FIGS. 3-4, and like elements are numbered accordingly. FIG. 5A illustrates the speaking valve 7 with its valve element 8 in its first position; FIG. 5B shows the valve element 8 in its second position and FIG. 5C shows the valve element 8 in its third position.

In this embodiment, the biasing element is an annular ring of a resilient open cell foam 28 located in the space between the valve body 9 and the valve element 8. The foam 28 is so sized as to be able to bias the valve element 8 into its fully open position as illustrated in FIG. 5C. One advantage of using an open cell foam is that, as well as performing a biasing function, it can also act as a heat and moisture reservoir, to help keep a user's trachea hydrated and warm.

FIG. 6 illustrates an embodiment of a speaking valve 7 of the invention in cross-sectional view to show the positioning of an alternative biasing element. The illustrated embodiment is the same as that illustrated in FIGS. 3-4, and like elements are numbered accordingly. In this embodiment, the biasing element comprises a helical spring 29 located in the space between the valve body 9 and the valve element 8. Open cell foam could also be added inside the helical spring to provide a moisture and heat reservoir if required.

1. A speaking valve to allow inspiration and controllable expiration through a tracheostoma, said speaking valve comprising:
a valve element movable by finger pressure of a user between a first position in which said speaking valve allows air to pass through it and a second position in which the passage of air through said speaking valve is substantially resisted or prevented;
a biasing element to bias said valve element towards said first position when no finger pressure is applied;
wherein said valve element is arranged such that expiration through the valve at a rate in excess of predetermined rate causes said valve element to move into a third position in which air resistance through the valve is less than when the valve element is in its first position.

2. A speaking valve according to claim 1 in which when said valve element is in its third position it can only be moved to its first position by finger pressure of a user.

3. A speaking valve according to either of claim 1 wherein said biasing element comprises resilient open cell foam.

4. A speaking valve according to either of claim 2 wherein said biasing element comprises resilient open cell foam.
